(19) United States
(12) Patent Application Publication
(10) Pub. No.: US 2025/0163289 A1
(43) Pub. Date: May 22, 2025

(54) BIOMEDICAL IMPLANT SYSTEM

(71) Applicant: KING FAHD UNIVERSITY OF PETROLEUM AND MINERALS, Dhahran (SA)

(72) Inventors: A. Madhan KUMAR, Dhahran (SA); Mohamed Abdrabou HUSSEIN, Dhahran (SA)

(73) Assignee: KING FAHD UNIVERSITY OF PETROLEUM AND MINERALS, Dhahran (SA)

(21) Appl. No.: 18/517,732

(22) Filed: Nov. 22, 2023

(30) Foreign Application Priority Data
Nov. 16, 2023 (SA) 123450838

(51) Int. Cl. C09D 127/16; A61M 37/00; C08K 3/22; C09D 5/14; C09D 7/61

(52) U.S. Cl. CPC C09D 127/16; A61M 37/00; C08K 3/22; C09D 5/14; C09D 7/61; C08K 2003/2241; C08K 2201/011

(57) ABSTRACT

A biomedical implant system includes a substrate and a coating. The coating includes a first layer including titanium dioxide nanotubes and a second layer including a polyvinylidene fluoride polymer. The titanium dioxide nanotubes extend perpendicularly from a surface of the substrate and contain ciprofloxacin. The titanium dioxide nanotubes are capped on an outer end with the polyvinylidene fluoride polymer. A process for making the biomedical implant system includes anodizing the substrate, at a voltage of 20 V to 80 V for a time of 10 to 200 minutes, in a solution to form the first layer of the coating. The first layer of the coating is further dried, annealed, loaded with ciprofloxacin, and covered with the second layer of the coating.

### CROSS REFERENCE TO RELATED APPLICATION

The present disclosure claims the benefit of Saudi patent application Ser. No. 12/345,0838 filed on Nov. 16, 2023, with the Saudi Authority for Intellectual Property Office, which is incorporated herein by reference in its entirety.

### STATEMENT OF ACKNOWLEDGEMENT

Support provided by King Fahd University of Petroleum and Minerals, Saudi Arabia, through Project ER221010 is gratefully acknowledged.

### BACKGROUND

### Technical Field

The present disclosure is directed to a biomedical implant, and more particularly, to a polymer-coated titanium-based biomedical implant system.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

With the advent of biotechnical and medical sciences, the consumption and implantation of biomedical orthopedic devices has increased over the past decades. The assessed market share of orthopedic devices is rising worldwide, and the requirement for total knee and hip implants is estimated to be about 3.48 million by 2030. As such, the development of bioimplants would be beneficial for both overall health and the economy. Traditional bioimplants come with drawbacks, such as infection at the implant site, injury or damage to surrounding structures, and/or nerve damage, which may result in chronic pain, numbness, or tingling in natural bones, joints, teeth, or gums. As medical science has progressed, titanium (Ti) and its alloy have become popular and frequently utilized materials in various biomedical segments. Areas such as orthopedics, implants, and dentistry have used titanium and titanium alloy materials due to their corrosion resistance, relatively low density, and low modulus of elasticity. Ti implants may fail for several reasons that ultimately lead to revision procedures, inducing heavy financial and mental burden on patients and recipients. One of the issues for Ti alloys is that Ti implants do not interact with the adjacent bone(s) in the first stages after implant surgery. Bacterial infection, tissue inflammation, and released metal ions due to corrosion are the leading origins of premature failure of Ti orthopedic implants. Due to added risks and lack of risk management, traditional implant procedures are avoided to an extent.

During recent decades, a drug delivery approach with a controlled release strategy has been used in implants to discourage infection at the site of the implant. The implants are covered with a biocompatible polymer to provide a slow release of the drug and lead to a more effective implant. The biocompatible polymer aids in the longevity of the implant by increasing the stability and drug loading capability of the implant. Polymer-based drug delivery models such as micelles, microspheres, polymer nanocomposites, hydrogels, and the like have been studied. Drugs and drug fragments are released through the polymeric film to the surrounding areas via erosion, diffusion, degradation of the film, and various other mechanisms.

Titanium nanotube (TNT) arrays have large surface areas, one-end open dimensions, high biocompatibility, and a capacity to regulate antibiotics and proteins. Although many biomedical implant systems have been developed in the past, there still exists a need to develop biomedical implant system with improved drug delivery and corrosion resistance.

CN102764599B discloses a method for the preparation of nano-material mixed substrate film, comprising two steps: step one, preparing a nanometer material with microporous layer; step two, implanting the nanomaterial having a microporous surface as a filter layer. The nanometer material film has high firmness and is uniformly distributed on the surface. However, assembly is not easy because the coverage of the film surface is not easy to control.

CN104826159A discloses a medical titanium metal implant material and the preparation method thereof. The medical titanium metal implant material comprises a titanium metal and a matrix of titanium dioxide nanotubes. The titanium dioxide nanotubes are covered with a degradable high polymer that covers an antibacterial antiphlogistic medicine in the titanium dioxide nanometer tube.

Accordingly, an object of the present disclosure is to provide methods and techniques for a biomedical implant system capable of delivering drugs sustainably and safely to implant sites.

### SUMMARY

In an exemplary embodiment, a biomedical implant system is described. The biomedical implant system includes a substrate and a coating. The coating includes a first layer, the first layer includes titanium dioxide nanotubes. The titanium dioxide nanotubes extend perpendicularly from a surface of the substrate and the titanium dioxide nanotubes contain ciprofloxacin. Further, the coating includes a second layer including a polyvinylidene fluoride polymer and the titanium dioxide nanotubes are capped on an outer end with the polyvinylidene fluoride polymer.

In some embodiments, the first layer consists of the titanium dioxide nanotubes aligned adjacently.

In some embodiments, a first end of the titanium dioxide nanotubes is attached to the surface of the substrate and a second end of the titanium dioxide nanotubes is open-faced and exposed to the polyvinylidene fluoride polymer.

In some embodiments, the titanium dioxide nanotubes have an inside diameter of 20 to 60 nanometers (nm).

In some embodiments, the titanium dioxide nanotubes have a length of 1 to 5 micrometers (μm).

In some embodiments, the titanium dioxide nanotubes have a wall thickness of 0.2 to 5 nm.

In some embodiments, the second layer has a thickness of 5 to 10 μm.

In some embodiments, the second layer has a thickness of 5 μm to 10 μm and is formed of a mixture of polyvinylidene fluoride and polylactic acid in a mass ratio of 70-95:5-30 such that up to 10% by number of the titanium dioxide nanotubes are capped on an outer end with the polylactic acid and the remaining titanium dioxide nanotubes are capped on an outer end with polyvinylidene fluoride polymer.

In some embodiments, the substrate is a commercially pure titanium sample.

In an exemplary embodiment, a method for preparing the biomedical implant system is also described. The method includes anodizing the substrate in a solution to form the first layer of the coating. The solution includes an organic solvent, water, and an inorganic compound. The anodizing is carried out at a voltage of 20 to 80 volts (V) for a time of 10 to 200 minutes. The method further includes drying the first layer of the coating, annealing the first layer of the coating, loading the first layer of the coating with ciprofloxacin, and covering the first layer of the coating with the second layer of the coating.

In some embodiments, the anodizing occurs in a two-electrode cell with the substrate for an anode and a graphite rod for a cathode.

In some embodiments, the loading is done in a polar organic solvent.

In some embodiments, the ciprofloxacin is loaded into the titanium dioxide nanotubes at a concentration of 0.01 to 0.10 molar (M).

In some embodiments, the biomedical implant system includes titanium, oxygen, carbon, nitrogen, and fluorine.

In some embodiments, 70 to 95 percent of titanium oxide in the titanium dioxide nanotubes have an anatase phase crystallinity.

In some embodiments, the biomedical implant system has a lower corrosion current compared to a polyvinylidene fluoride polymer film-coated titanium sample and a polyvinylidene fluoride polymer film-coated titanium dioxide nanotube sample in the absence of ciprofloxacin. In some embodiments, the biomedical implant system has a greater antibacterial resistance to Escherichia coli compared to a bare titanium sample, a polyvinylidene fluoride polymer film-coated titanium sample, and a polyvinylidene fluoride polymer film-coated titanium dioxide nanotube sample in the absence of ciprofloxacin.

In some embodiments, a rate of antibacterial activity is from 97 to 99 percent based on an area of microbial growth inhibition.

In some embodiments, the biomedical implant system has a greater antibacterial resistance to Staphylococcus aureus compared to a bare titanium sample, a polyvinylidene fluoride polymer film-coated titanium sample, and a polyvinylidene fluoride polymer film-coated titanium dioxide nanotube sample in the absence of ciprofloxacin.

In some embodiments, the ciprofloxacin is released through the polyvinylidene fluoride polymer over time.

These and other aspects of non-limiting embodiments of the present disclosure will become apparent to those skilled in the art upon review of the following description of specific non-limiting embodiments of the disclosure in conjunction with the accompanying drawings. The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure (including alternatives and/or variations thereof) and many of the attendant advantages thereof will be readily obtained as the same becomes better understood by reference to the following detailed description when considered in connection with the accompanying drawings, wherein:

FIG. 1A is a schematic flow chart diagram of a method of the fabrication of the biomedical implant system, according to certain embodiments;

FIG. 1B is a schematic process flow chart illustration of a method of coating and activating the biomedical implant system, according to certain embodiments;

FIG. 2A is a field emission scanning electron microscopy (FESEM) image at 120,000× of titanium dioxide nanotubes at a potential difference of 40 volts (V), according to certain embodiments;

FIG. 2B is an FESEM image at 120,000× of titanium dioxide nanotubes at a potential difference of 50 V, according to certain embodiments;

FIG. 2C is an FESEM image at 120,000× of titanium dioxide nanotubes at a potential difference of 60 V, according to certain embodiments;

FIG. 2D is an FESEM image at 240,000× of titanium dioxide nanotubes at a potential difference of 50 V, according to certain embodiments;

FIGS. 3A-3B shows a scanning electron microscopy (SEM) image of the titanium dioxide nanotubes fabricated on commercially pure titanium using anodization, according to certain embodiments;

FIG. 3C shows energy-dispersive X-ray (EDX) spectroscopy of the titanium dioxide nanotubes fabricated on commercially pure titanium, according to certain embodiments;

FIG. 3D shows EDX layered image of elemental mapping of the titanium dioxide nanotubes fabricated on commercially pure titanium, according to certain embodiments;

FIG. 3E depicts titanium elemental mapping data of the titanium dioxide nanotubes fabricated on commercially pure titanium, obtained via EDX analysis overlaid onto the SEM image, according to certain embodiments;

FIG. 3F depicts oxygen elemental mapping data of the titanium dioxide nanotubes fabricated on commercially pure titanium, obtained via EDX analysis overlaid onto the SEM image, according to certain embodiments;

FIG. 4 depicts a potentiodynamic polarization (PDP) curve of the fabricated titanium dioxide nanotubes in simulated body fluids, according to certain embodiments;

FIG. 5A shows an X-ray diffraction (XRD) pattern of bare titanium, according to certain embodiments;

FIG. 5B depicts an XRD phase pattern of the anodized titanium dioxide nanotubes at 40 V, according to certain embodiments;

FIG. 5C depicts an XRD phase pattern of the anodized titanium dioxide nanotubes at 50 V, according to certain embodiments;

FIG. 5D depicts an XRD phase pattern of the anodized titanium dioxide nanotubes at 60 V, according to certain embodiments;

FIG. 5E depicts an XRD pattern of titanium dioxide nanotubes with polyvinylidene fluoride (PVDF) at 50 V, according to certain embodiments;

FIG. 5F depicts XRD analysis of titanium oxide and anatase and rutile phases of titanium dioxide, according to certain embodiments;

FIG. 6A is a scanning electron microscopy of PVDF film deposited on drug-loaded titanium dioxide nanotubes, according to certain embodiments;

FIG. 6B is an EDX spectrum of the PVDF film deposited on the drug-loaded titanium dioxide nanotubes, according to certain embodiments;

FIG. 6C is an EDX layered image of PVDF film deposited on the drug-loaded titanium dioxide nanotubes, according to certain embodiments;

FIGS. 6D-6H shows EDS elemental mapping images for titanium, oxygen, carbon, nitrogen, and fluorine, respectively, according to certain embodiments;

FIG. 7 depicts PDP curves of PVDF films deposited on drug-loaded titanium dioxide nanotube substrates in simulated body fluid (SBF) medium, according to certain embodiments;

FIG. 8A shows antibacterial evaluation of PVDF-coated titanium dioxide nanotube loaded with antibacterial drug for gram-positive bacteria, according to certain embodiments; and

FIG. 8B shows antibacterial evaluation of PVDF-coated titanium dioxide nanotubes loaded with antibacterial drug for gram-negative bacteria, according to certain embodiments.

### DETAILED DESCRIPTION

In the following description, it is understood that other embodiments may be utilized, and structural and operational changes may be made without departure from the scope of the present embodiments disclosed herein.

Reference will now be made in detail to specific embodiments or features, examples of which are illustrated in the accompanying drawings. Whenever possible, corresponding, or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may be construed to relate to the plural and vice-versa without limiting the scope of the disclosure to the exact number or type of such elements unless set forth explicitly in the appended claims. In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of this disclosure are directed towards a biomedical implant and drug delivery system and a method thereof. The biomedical implant includes a commercially pure titanium substrate coated with a polymeric matrix/a coating from a combination of a biopolymer (polyvinylidene fluoride (PVDF)) and an antibiotic drug (ciprofloxacin) loaded into Ti nanotubes (TNT) for biomedical applications. The structure, microstructure, and surface properties of the TNT surface and the polymer-coated TNT surface are characterized using X-ray diffraction (XRD), scanning electron microscopy/energy dispersive spectroscopy (SEM/EDS), Fourier-transform infrared (FTIR) spectroscopy, and contact angle measurement techniques. The performance of the bioimplant was evaluated by studying the in vitro corrosion protection in simulated body fluid (SBF) and antibacterial efficiency. The test outcomes supported the sustained release of the antibacterial drug, ciprofloxacin, from the polymer matrix, providing effective antibacterial performance against gram-positive and gram-negative bacterial systems. The polymer matrix coating enhanced the in vitro corrosion resistance performance of Ti substrates in the SBF.

A biomedical implant system is described. In some embodiments, the substrate may be an orthopedic implant. In an embodiment, the biomedical implant may be bone screws, such as pedicle screws and fixation screws, cylinder implants, blade implants, mandibular implants, dental implants, hip screws, shaped bone prosthetics, plates, rods, hip replacement parts, knee replacement parts, shoulder replacement parts, elbow replacement parts, fusion cages and all other types of implants for use at or near the bone. In an embodiment, the biomedical implant may be any implantable medical device. In an embodiment, the biomedical implant may be at least partially placed within a patient's body. In some embodiments, the biomedical implant, also referred to herein as bioimplant or implant, may be placed within the patient's body permanently or for a period of time for which it may be beneficial to have a therapeutic agent present. In another embodiment, the implant may be a stent including, but not limited to, arterial, esophageal, biliary, colon, urethral, airway, and lacrimal stents. The stent, for example, may be a balloon expandable stent, self-expandable stent, tubular stent, or coil stent. In some embodiments, the implant may be any biocompatible implant. In a preferred embodiment, the implant may be an implantable drug repository used to deliver biological agents, such as pharmaceuticals, inside the body.

The biomedical implant system is herein referred to as a system for the sake of brevity. The system includes a substrate coated with a coating. The substrate refers to a substance or a surface on which further developmental modifications can be made. A metal, ceramic, or plastic material may be used as the substrate of the bioimplant. A stainless-steel alloy, a cobalt-chromium alloy, titanium, a titanium alloy, titanium-based amorphous alloys, alumina, zirconia, or the like may be used as the substrate. In a preferred embodiment, the substrate is titanium and/or titanium alloys. The titanium alloys for use include alloys of titanium with at least one metal selected from aluminum, tin, zirconium, molybdenum, nickel, palladium, tantalum, niobium, vanadium, platinum, and the like. One such titanium alloy commonly used in orthopedic applications because of its strength, corrosion resistance, and biocompatibility is Ti-6Al-4V ELI (extra low interstitial), comprising of titanium, aluminum, and vanadium. Titanium and/or its alloys may be generally favored as substrates because titanium is a bioinert material that facilitates osseointegration. In a preferred embodiment, the substrate is a commercially pure (“CP”) titanium sample. The substrate may be in the form of a rod, a sheet, a plate, a screw, a ribbon, a wire, a mesh, a foam, a sphere, a cube, a rectangle, a shape, or the like. In an embodiment, the substrate is an orthopedic implant.

The surface of the substrate is coated, at least partially, about 50%, preferably about 60%, preferably about 70%, preferably about 80%, preferably about 85%, preferably about 90%, preferably about 95%, preferably about 97%, more preferably about 99%, and yet more preferably about 100%, with a coating. The coating includes a plurality of layers. In a specific embodiment, the coating includes two layers-a first layer and a second layer. Each of the layers, i.e., the first layer and the second layer, may include one or more sub-layers. The first layer includes and/or consists of titanium dioxide nanotubes. The titanium dioxide nanotubes extend perpendicularly from the surface of the substrate. The titanium dioxide nanotubes may extend at an angle of 60 to 120°, preferably 70 to 110°, preferably 80 to 100°, or preferably about 90° from the surface of the substrate.

In some embodiments, the titanium dioxide (TiO₂) in the titanium dioxide nanotubes are 70% to 95%, preferably 75% to 90%, or preferably 80% to 85%, have an anatase phase crystallinity. The titanium dioxide in the titanium dioxide nanotubes may be more than about 5%, about 15%, about 25%, about 35%, about 45%, about 55%, about 65%, about 75%, about 85%, and about 95% in the anatase phase and may include, in minor amounts, a rutile phase. The anatase phase titanium dioxide can be described as a cohesive single-phase oxide that exhibits a distinct crystallographic X-ray structure. The anatase phase is a metastable mineral form of TiO₂ with a tetragonal crystal structure.

The first layer consists of the titanium dioxide nanotubes aligned adjacently. The titanium dioxide nanotubes have a diameter between 30 nm to 80 nm, preferably 35 to 70 nm, preferably 20 to 60 nm. The titanium dioxide nanotubes have an inner diameter of 20 to 60 nm, preferably 25 to 55 nm, more preferably 30 to 50 nm, and yet more preferably about 35 to 45 nm. In an embodiment, the titanium dioxide nanotubes have a wall thickness of 0.2 to 5 nm, preferably 0.5 to 3 nm, and preferably 1 to 2 nm. The titanium dioxide nanotubes have a length of 1 to 5 μm, preferably 2 to 4 μm, and more preferably about 3 to 4 μm.

In an embodiment, the titanium dioxide nanotubes contain ciprofloxacin. Although the examples and the description herein provided refer to the use of ciprofloxacin in the first layer, it may be understood by a person skilled in the art, that with minor chemical, thermal, or mechanical modifications, the bioimplant may be adapted to include other pharmaceuticals as well. Suitable examples of other pharmaceuticals include, but are not limited to, analgesics such as codeine, morphine, ketorolac, naproxen, anesthetics, anti-inflammatory agents, antibacterials, antiviral drugs such as acyclovir, gancyclovir, vidarabine, azidothymidine, dideoxyinosine, dideoxycytosine, antifungals such as amphotericin, antiprotozoals, anti-infectives, antibiotics, aminoglycosides such as gentamicin, kanamycin, tobramycin, plazomicin, streptomycin, neomycin, and vancomycin, amphenicols such as chloramphenicol cephalosporins, such as cefazolin HCl, penicillins such as ampicillin, penicillin, carbenicillin, oxycillin, methicillin, lincosamides such as lincomycin, polypeptide antibiotics such as polymixin and bacitracin, tetracyclines such as tetracycline, minocycline, and doxycycline, quinolones such as ciprofloxacin, moxifloxacin, gatifloxacin, and levofloxacin, lidocaine, cannabinoids, anti-angiogenesis compounds such as anecortave acetate, retinoids such as tazarotene, steroidal anti-inflammatory agents such as 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and/or combinations thereof, as well as any drugs known in the art. In a preferred embodiment, the pharmaceutical is ciprofloxacin.

The titanium dioxide nanotubes extend perpendicularly from the surface of the substrate, and each nanotube is aligned adjacently aligned to another nanotube. The titanium dioxide nanotubes may exist as bunches or bundles of tubes. The titanium dioxide nanotubes of the first layer may be bordering 2 to 12, preferably 4 to 10, or preferably 6 to 8 other titanium dioxide nanotubes of the first layer. In an embodiment, an opening of the titanium dioxide nanotubes is in the form of a circle. Although the description refers to titanium dioxide nanotubes, nanorods may be present as well. The titanium dioxide nanotubes may be flat or cylindrical in shape. In a preferred embodiment, the titanium dioxide nanotubes are cylindrical. The titanium dioxide nanotubes include two ends, a first end and a second end. The first end of the titanium dioxide nanotubes is attached to the surface of the substrate and the second end of the titanium dioxide nanotubes is open-faced and exposed to the second layer of the coating.

The second layer includes a polymer matrix/polymeric component/polymer. The polymeric component may enhance the drug's pharmacokinetic features, increase its therapeutic index, decrease its harmful effects, and improve the overall effectiveness of biomedical implants. In addition, the role of polymers in framing a drug delivery system has been considered due to their stability, drug-loading capability, and adapted characteristics. Particularly, biocompatible and biodegradable polymers are used for drug delivery application due to the requisite of suitable discharge of the drug and easy elimination of the carrier from the physiological environment. In some embodiments, the second layer may include one or more polymers selected from collagen, gelatin, chitosan, and/or synthetic polymers such as poly(lactic acid) (PLA), poly(glycolic acid) (PGA), polycaprolactone (PCL), and PVDF-hydroxyapatite composites. In some embodiments, the second layer may optionally include polymeric precursors of polymers selected from poly(phosphoesters), polysulfones, polyfumarates, polyphosphazines, poly(alkylene oxides), poly(arylates), poly(anhydrides), poly(hydroxy acids), polyesters, poly(ortho esters), polycarbonates, poly(propylene fumerates), poly(caprolactones), polyamides, polyamino acids, polyacetals, polylactides, polyglycolides, poly(dioxanones), polyhydroxybutyrate, polyhydroxyvalyrate, poly(vinyl pyrrolidone), biodegradable polycyanoacrylates, biodegradable polyurethanes, polysaccharides, tyrosine-based polymers, poly(pyrrole), poly(aniline), poly(thiophene), polystyrene, non-biodegradable polyurethanes, polyureas, poly(ethylene vinyl acetate), polypropylene, polymethacrylate, polyethylene, poly(ethylene oxide), polyvinylidene fluoride, and mixtures, adducts, derivatives, and co-polymers thereof. In a specific embodiment, the second layer comprises a polyvinylidene fluoride (PVDF) polymer.

The titanium dioxide nanotubes are capped on an outer end with the PVDF polymer. The second layer has a thickness of 5 to 10 μm, preferably 6 to 9 μm, or preferably 7 to 8 μm. The first layer and the second layer together have a thickness in a range of 1 to 20 μm preferably 2 to 18 μm, preferably 3 to 16 μm, preferably 4 to 14 μm, preferably 5 to 12 μm, preferably 6 to 10 μm, preferably 7 to 8 μm, and yet more preferably about 7.5 μm.

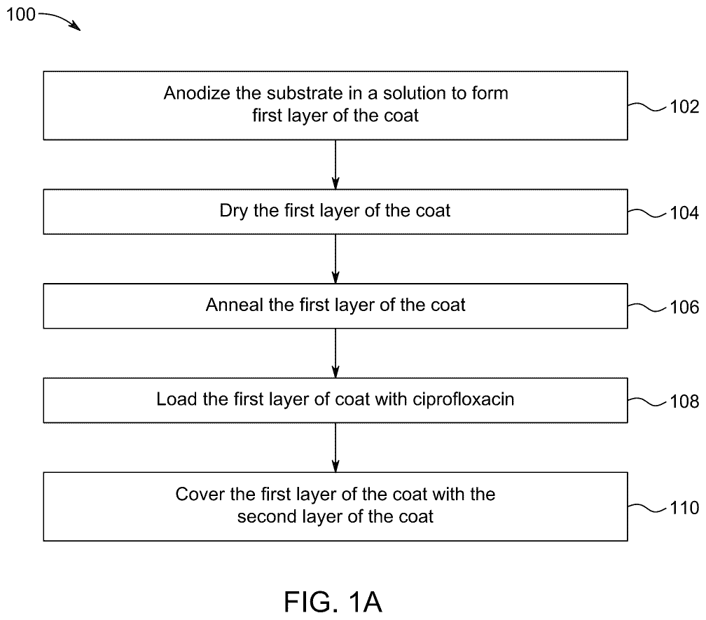

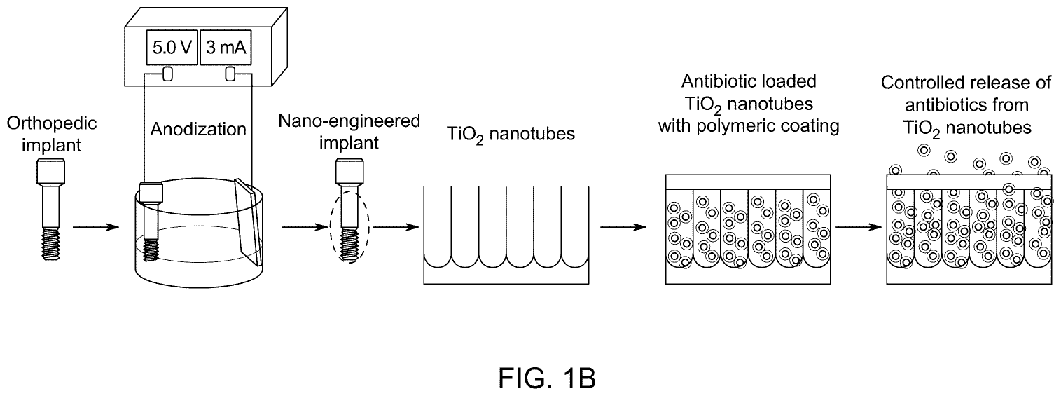

Referring to FIG. 1A, a flow chart diagram of a method 100 for fabrication of the biomedical implant system is illustrated. The order in which the method 100 is described is not intended to be construed as a limitation, and any number of the described method steps may be combined in any order to implement the method 100. Additionally, individual steps may be removed or skipped from the method 100 without departing from the spirit and scope of the present disclosure.

At step 102, the method 100 includes anodizing the substrate in a solution to form the first layer of the coating. Anodizing is an electrolytic passivation process used to increase the thickness of the natural oxide layer on the surface of metal parts. In a specific embodiment, the anodizing is carried out by an electrolytic process. The electrolytic process for anodizing occurs in a two-electrode cell. The substrate forms the anode, while platinum, gold, or carbon serves as the cathode. The carbon may be in the form of graphite or glassy carbon. In a preferred embodiment, a graphite rod serves as the cathode. In a particular embodiment, the commercially pure titanium substrate serves as an anode and a graphite rod is the cathode. The commercially pure titanium has a length of 2 cm, a width of 2 cm, and a thickness of 0.3 cm. The solution comprises an organic solvent, water, and an inorganic compound. The organic solvent is ethylene glycol. The inorganic compound may be a fluoride, for example, hydrogen fluoride or ammonium fluoride (NH₄F), to increase the compatibility of the titanium dioxide nanotubes with the substrate. In a preferred embodiment, the inorganic compound is NH₄F. The anodization may be carried out at a voltage in the range of 20 to 80 V, preferably 40 to 60 V, for a time of 10 to 200 minutes, preferably 30 to 90 minutes. In some embodiments, prior to the anodizing process, the substrate may be subjected to surface preparation by acid/alkaline pre-treatment to make the substrate more suitable to fabricate titanium dioxide nanotubes thereupon.

At step 104, the method 100 includes drying the first layer of the coating. The drying can be done by using heating appliances such as ovens, microwaves, autoclaves, hot plates, heating mantles and tapes, oil baths, salt baths, sand baths, air baths, hot-tube furnaces, hot-air guns, hot air blowers, a combination thereof, and the like. In an embodiment, the drying may be carried out by a hot air blower to prevent the formation of an additional layer thereupon.

At step 106, the method 100 includes annealing the first layer of the coating. Titanium dioxide nanotubes formed on the surface of the substrate are rinsed with distilled water and further dried by annealing at a temperature of 200 to 700° C., preferably 300 to 600° C., more preferably 400 to 500° C., and yet more preferably at a temperature of about 450° C. for 0.5 to 10 hours, preferably 1 to 5 hours, and more preferably for about 2 hours.

At step 108, the method 100 includes loading the first layer of the coating with ciprofloxacin. The loading is done in a polar organic solvent. The ciprofloxacin is loaded into the titanium dioxide nanotubes at a concentration of 0.01 to 0.10 M, preferably 0.03 to 0.07 M, preferably about 0.05 M. Suitable examples of the organic solvent include tetrahydrofuran, ethyl acetate, dimethylformamide (DMF), acetonitrile, acetone, dimethyl sulfoxide, nitromethane, propylene carbonate, ethanol, formic acid, n-butanol, methanol, benzene, cyclohexane, ethyl acetate, dichloromethane, toluene, diethyl ether, and the like or any combination thereof. In a preferred embodiment, the organic solvent is ethanol. The ciprofloxacin may be loaded into the titanium dioxide nanotubes in a dropwise manner. The ciprofloxacin may be loaded into the titanium dioxide nanotubes in 1 to 5 drops, preferably 2 to 4 drops, and preferably about 3 drops. The ciprofloxacin may be loaded into the titanium dioxide nanotubes in 1 to 5 cycles, preferably, 2 to 4 cycles, and more preferably 3 cycles.

At step 110, the method 100 includes covering the first layer of the coating with the second layer of the coating. The second layer includes one or more polymers. Suitable examples of polymers include collagen, gelatin, chitosan, and synthetic polymers such as poly(lactic acid) (PLA), poly(glycolic acid) (PGA), polycaprolactone (PCL), and PVDF-hydroxyapatite composites. In a preferred embodiment, the second layer includes PVDF. The second layer is formed by dissolving a polymer, preferably PVDF, in an organic solvent at a temperature range of 80 to 120° C., preferably 90 to 110° C., and preferably about 100° C. In a preferred embodiment, the organic solvent the polymer is dissolved in is DMF. The substrate coated with ciprofloxacin-loaded titanium dioxide nanotubes is dipped in the second layer for 8 to 12 seconds, preferably 9 to 11 seconds, and preferably about 10 seconds, and further dried to evaporate the organic solvent. The substrate coated with ciprofloxacin-loaded titanium dioxide nanotubes is dipped for 2 to 10 cycles, preferably 3 to 8 cycles, preferably 4 to 6 cycles, and preferably about 5 cycles. A time of 60 to 300 seconds, preferably 90 to 180 seconds, and preferably about 120 seconds, is waited in between the cycles. The PVDF film deposited on drug-loaded titanium dioxide nanotubes was dried at a temperature range of 150 to 200° C., preferably 160 to 190° C., preferably about 180° C. to fabricate the biomedical implant.

In a preferred environment, the polymer is a blend or mixture of two immiscible polymers. Preferably, a mixture comprising PVDF and a second polymer such as poly lactic acid (e.g., PLLA). The deposition of this mixture of immiscible polymers forms an irregular film coating over the open ends of the titanium dioxide nanotubes. The resultant polymer layer has a sea-island structure with the islands representing the polymer present in a lower amount by mass in the blend of immiscible polymers. The islands are preferably homogeneously dispersed over the titanium dioxide nanotube layer, for example, the islands having a largest diameter of 50-1,000 nm, preferably 200-400 nm. Preferably, PVDF is the main component of the immiscible polymer blend. Most of the open ends of the titanium dioxide nanotubes are thus capped with a PVDF polymer. A fraction of the open ends of the tubes are capped primarily with the second polymer of the immiscible polymer blend. This second capping is preferably from 1 to 10% by number of open ends of titanium dioxide nanotubes per area, preferably 2 to 8%, 3 to 5%, or about 4% by number. The presence of two dissimilar and immiscible polymers as capping agents for the titanium dioxide nanotubes provides different release rates and release conditions for the material inside the titanium dioxide nanotubes. This, in turn, permits adjusting the release rate of, for example, ciprofloxacin.

The biomedical implant system comprises titanium, oxygen, carbon, nitrogen, and fluorine. The biomedical implant system prepared by the method of present disclosure has a lower corrosion current compared to a PVDF polymer film-coated titanium sample and a PVDF polymer film-coated titanium dioxide nanotube sample in the absence of ciprofloxacin. The biomedical implant system also has a greater antibacterial resistance against both gram-positive and gram-negative bacteria. The biomedical implant system has a greater antibacterial resistance to Escherichia coli compared to a bare titanium sample, a PVDF polymer film-coated titanium sample, and a PVDF polymer film-coated titanium dioxide nanotube sample in the absence of ciprofloxacin. The rate of antibacterial activity is from 97 to 99 percent based on an area of microbial growth inhibition. The biomedical implant system has a greater antibacterial resistance to Staphylococcus aureus compared to a bare titanium sample, a PVDF polymer film-coated titanium sample, and a PVDF polymer film-coated titanium dioxide nanotube sample in the absence of ciprofloxacin.

The ciprofloxacin is released through the PVDF polymer of the biomedical implant system over time. In an embodiment, the ciprofloxacin is released over hours, days, weeks, months, years, and decades. In some embodiments, the ciprofloxacin is released over 2 to 72 hours, preferably 6 to 60 hours, preferably 12 to 48 hours, preferably 24 to 36 hours. In other embodiments, the ciprofloxacin is released over 1 to 30 days, preferably 2 to 25 days, preferably 5 to 20 days, preferably 10 to 15 days. In some embodiments, the ciprofloxacin is released over 1 to 20 weeks, preferably 2 to 16 weeks, preferably 4 to 12 weeks, preferably 6 to 10 weeks. In an embodiment, the ciprofloxacin is released over 1 to 18 months, preferably 2 to 12 months, preferably 3 to 9 months. . . . In an embodiment, the ciprofloxacin is released over 1 to 20 years, preferably 2 to 15 years, preferably 3 to 12 years, preferably 4 to 10 years, preferably 5 to 8 years. In some embodiments, the ciprofloxacin is released over 1 to 6 decades, preferably 2 to 5 decades, preferably 3 to 4 decades. In an embodiment, the ciprofloxacin is released over any time suitable towards an application of the biomedical implant system.

### EXAMPLES

The following examples describe and demonstrate exemplary embodiments of a biomedical implant as described herein. The examples are provided solely for illustration and are not to be construed as limitations of the present disclosure, as many variations thereof are possible without departing from the spirit and scope of the present disclosure.

### Example 1: Materials

Commercially pure titanium (CP-Ti) was utilized as the base substrate with the size of 2 cm×2 cm×0.3 cm and, prior to coating, proper surface preparation was performed. Cp-Ti surface substrates were prepared by grinding the surfaces with silicon carbide (SiC) grit papers from a size of 400 grit to 2400 grit and polishing with alumina to achieve a mirror-like surface. To remove residuals after polishing, the CP-Ti substrates were cleaned with distilled water, ultrasonicated with acetone, and dried in air. After surface preparation, the surface was followed by an acid/alkaline pre-treatment to make the surface of the CP-Ti more suitable for fabrication of the titanium dioxide nanotubes (TNTs).

### Example 2: Fabrication of TNTs

Preliminary experiments were done to determine the anodization parameters for the fabrication of the TNTs on the CP-Ti substrates based on the dimension and durability of the TNTs. Electrochemical anodization used a two-electrode cell with the CP-Ti substrate as the anode and a graphite rod as a cathode. The electrolyte used for anodization is ethylene glycol (47.5 mL) containing 2.5 mL of H₂O and 0.25 g of ammonium fluoride (NH₄F). The anodization was performed under different applied voltages (40 V, 50 V, and 60 V) for three periods of time (30, 60, and 90 minutes). After anodization, the specimens were dried by a hot air blower to prevent the formation of an additional layer above the nanotubes. TNTs formed on the Ti surface were rinsed with distilled water and dried by annealing at 450° C. for two hours.

### Example 3: Incorporation of Antibacterial Drugs

An antibacterial drug solution, including 0.1 g of ciprofloxacin and 10 mL of ethanol, was loaded onto the TiO₂ nanotubes in three cycles. At each cycle, three drops of the antibacterial drug solution were added to the TiO₂ nanotubes. The TiO₂ nanotubes were dried and kept dry until the start of the next cycle.

### Example 4: Deposition of PVDF Coatings on Drug-Loaded TNT Surface

After the antibacterial loading, 1 g of PVDF powder was dispersed in 10 mL dimethyl formamide (DMF), which was stirred by magnetic stirring at 100° C. to get a homogenous solution. The polymer coating was performed for 5 cycles by a dip coating method. The TNTs were dipped in the homogeneous solution for 10 seconds during each cycle. Further, a waiting period of 120 seconds was observed before starting the next cycle. After coating, the CP-Ti substrate was subjected to a heat treatment in a furnace at 180° C. for 1 hour. A schematic process flow chart illustration depicting the method of coating and activating the biomedical implant system is depicted in FIG. 1B.

### Example 5: Structure, Microstructure, and Surface Analysis

The surface morphology of the fabricated TNTs and coated CP-Ti were analyzed using scanning electron microscopy (SEM) at an irradiation current of 10 μA and an acceleration voltage of 20 kV. Energy-dispersive X-ray spectroscopy (EDS) analysis was also used to confirm the homogeneous distribution of drugs in the polymer matrix. Further, attenuated total reflectance infrared (ATR-IR) spectra of the polymer composite coatings were obtained in the region of 400 to 4000 cm⁻¹ to determine the chemical structure of the synthesized coatings on the Ti specimens. Furthermore, UV-visible analysis in the range 200 to 800 nm was carried out for the prepared polymer coatings to demonstrate the interfacial interaction between polymer and loaded drug.

### Example 6: In-Vitro Corrosion Study in Simulated Body Fluid

The electrochemical corrosion performance of bare and coated CP-Ti specimens was analyzed using simulated body fluid (SBF) using potentiodynamic polarization (PDP) and electrochemical impedance spectroscopy (EIS) measurements. The Gamry Reference 3000 electrochemical instrument studied in vitro corrosion resistance in SBF. The specimen of CP-Ti with an exposed part of 1 cm² was a working electrode, the graphite plate was a counter electrode, and the saturated calomel electrode (SCE) was the reference electrode for the three-electrode configuration. The open circuit potential (OCP) was observed during studies for about 30 minutes to achieve an electrochemically steady state in the investigated systems. EIS measurements were carried out using the appropriate frequencies of 1 kHz to 1 MHz through a 10 mV/decade perturbation amplitude. PDP tests were directed by scanning the potential from-250 mV vs. OCP to 1500 mV vs. SCE at a scan rate of 0.1967 mV/s.

### Example 7: Antibacterial Tests

The antibacterial behavior of the coated CP-Ti specimens was estimated against gram-negative bacteria, E. coli, and gram-positive bacteria, S. aureus, utilizing the surface spread plate technique. Firstly, the specimens were washed with ethanol to eliminate pollutants and germs, and then immersed in 5 mL of sterile water. Secondly, the bacterial cell suspension was shaken at 37° C. at 100 rpm. The quantity of viable cells was assessed and altered according to the dilution factor.

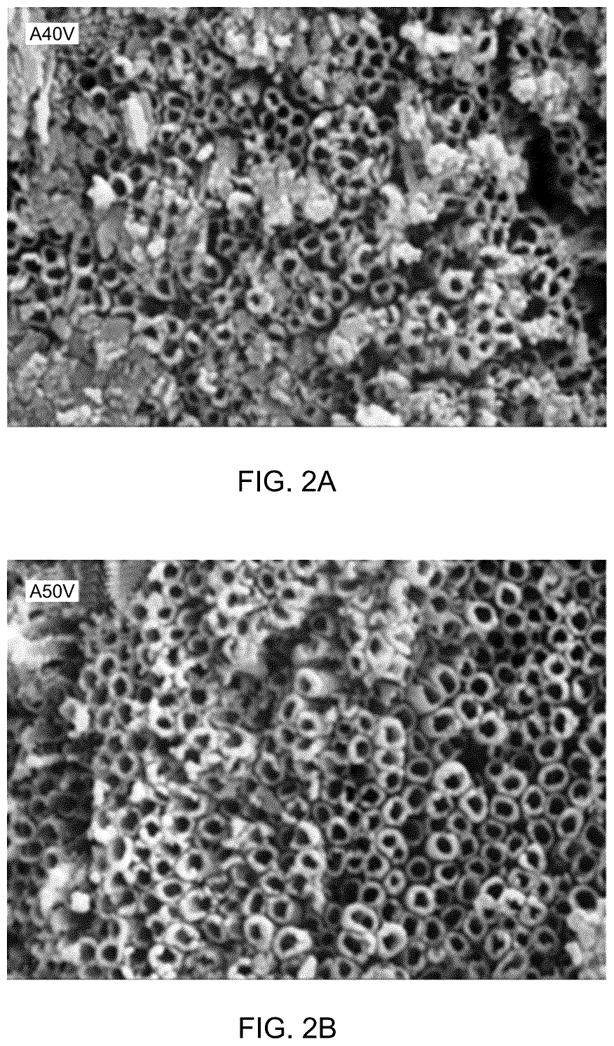

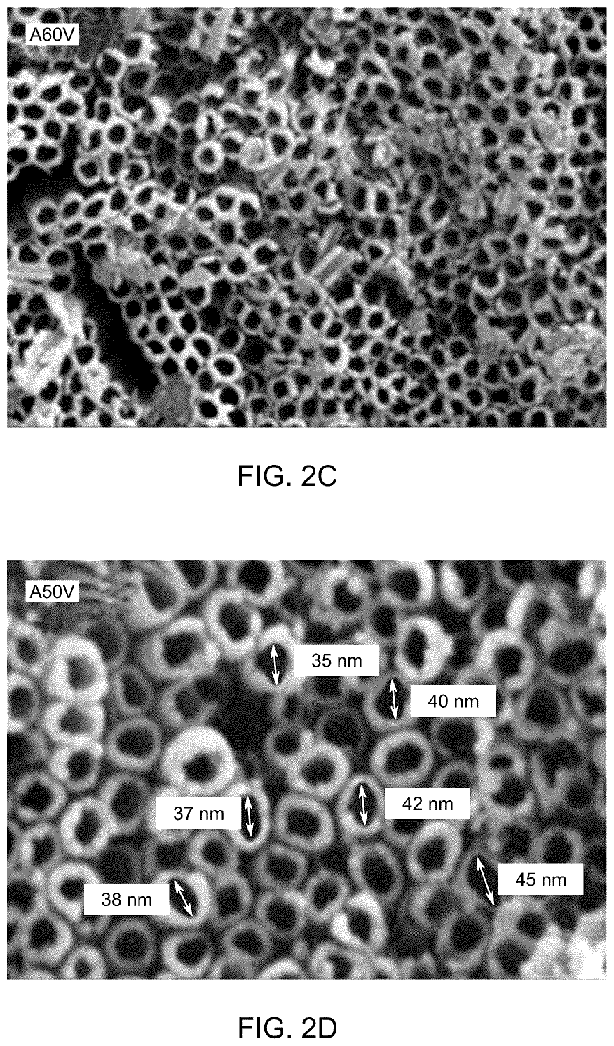

Referring to FIGS. 2A-2D, field emission scanning electron microscopy (FESEM) images of the titanium dioxide nanotube surface synthesized at a potential difference of 40 V, 50 V, and 60 V, respectively, are depicted. The formation and growth of the titanium dioxide nanotubes at different parameters, including applied voltages (potential differences) and time of anodization, were investigated to determine the appropriate potential difference according to the surface morphologies of fabricated titanium dioxide nanotubes and the corrosion resistance thereof. It was noted that the formation of pores on the titanium surface of the titanium dioxide nanotubes usually started at 20 V and above (without additive material). Below an applied potential of 20 V, small and weak tubes were formed. As such, the applied potential difference parameters were 40 V and above. The three applied voltages of 40 V, 50 V, and 60 V were evaluated with an anodization time of 30 minutes, 45 minutes, and 60 minutes. The preferred applied voltage of 50 V at 30 minutes (FIG. 2B), provided homogeneous titanium dioxide nanotubes, as well as improved the corrosion resistance of the substrate among the investigated samples. FIG. 2D depicts the diameter of the fabricated titanium dioxide nanotubes via a microscopic image. The inner diameter of the titanium dioxide nanotubes is in the range of 35 nm to 45 nm.

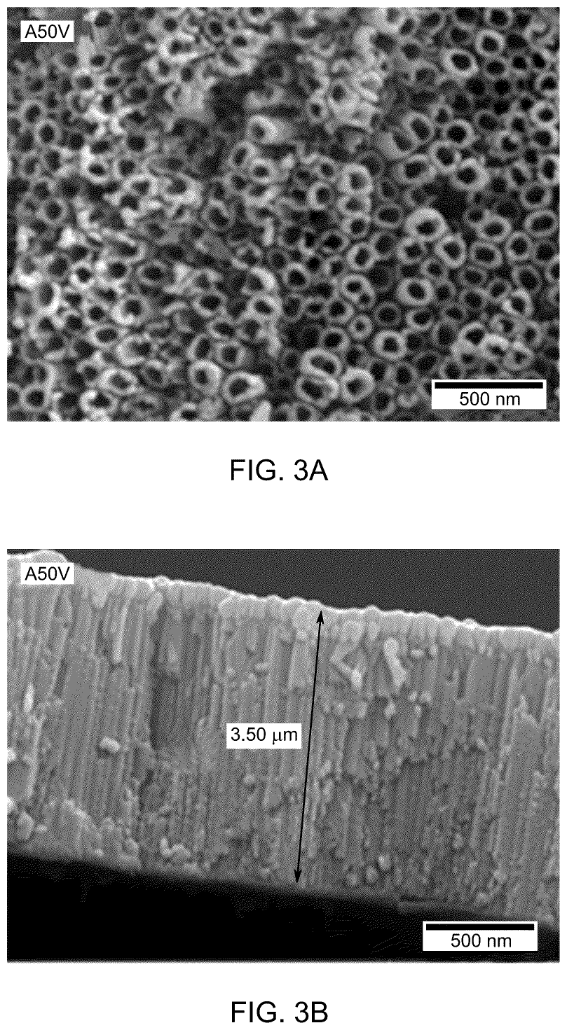

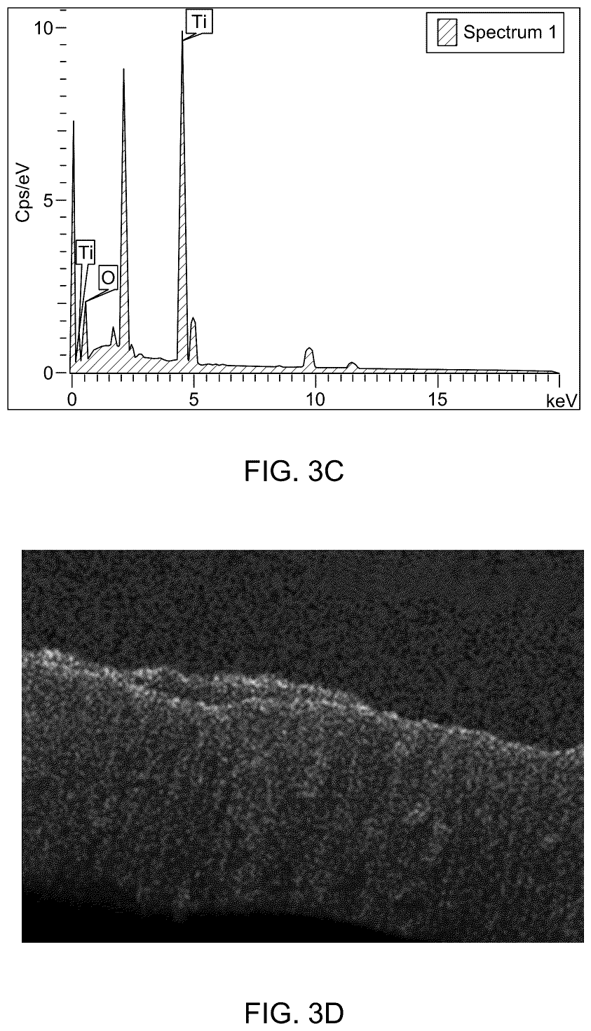

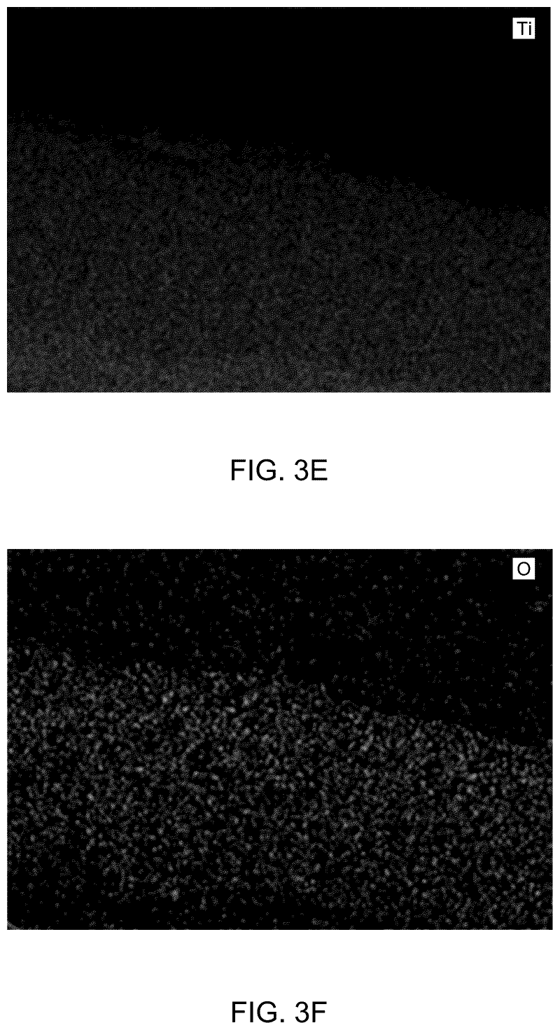

Referring to FIGS. 3A-3B, scanning electron microscopy (SEM) images depicting the diameter and length of the fabricated titanium dioxide nanotubes are illustrated. The titanium dioxide nanotubes formed at 50 V with an anodization time of 30 minutes exhibited a diameter of approximately 50 nm with a length of approximately 3.50 μm. Referring to FIGS. 3C-3D, EDS analysis and EDS mapping results are illustrated. As can be seen from FIGS. 3C-3D, EDS mapping results confirm and support that the fabricated titanium dioxide nanotubes include titanium peaks and oxide peaks, further validating and indicating the purity of the fabricated titanium dioxide nanotubes. Referring to FIGS. 3E-3F, elemental mapping data of the titanium dioxide nanotubes fabricated on commercially pure titanium, obtained via EDX analysis, is overlaid onto the SEM image. The elemental mapping data reveals the presence of titanium (FIG. 3E) and oxygen (FIG. 3F).

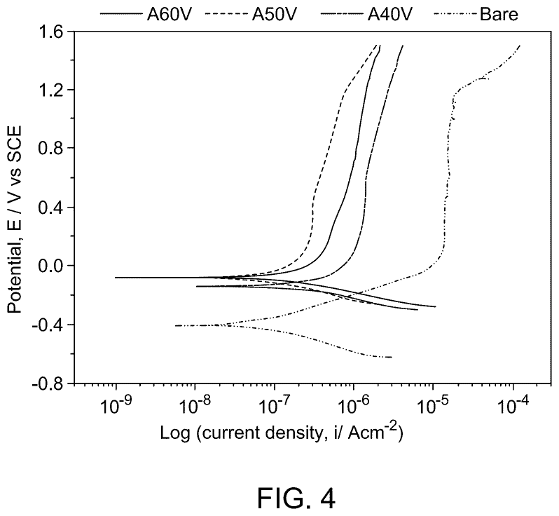

Referring to FIG. 4, a graph depicting the PDP curve of the fabricated titanium dioxide nanotubes in SBF is illustrated. Based on the obtained electrochemical corrosion test results, the titanium dioxide nanotubes formed at 50 V provided the highest corrosion-resistant behavior. It was compared to other titanium samples and documented by showing the nobler shift in the corrosion potentials and reduction in corrosion current density values of bare titanium, CP-Ti at 40 V, CP-Ti at 50 V, and CP-Ti at 60 V.

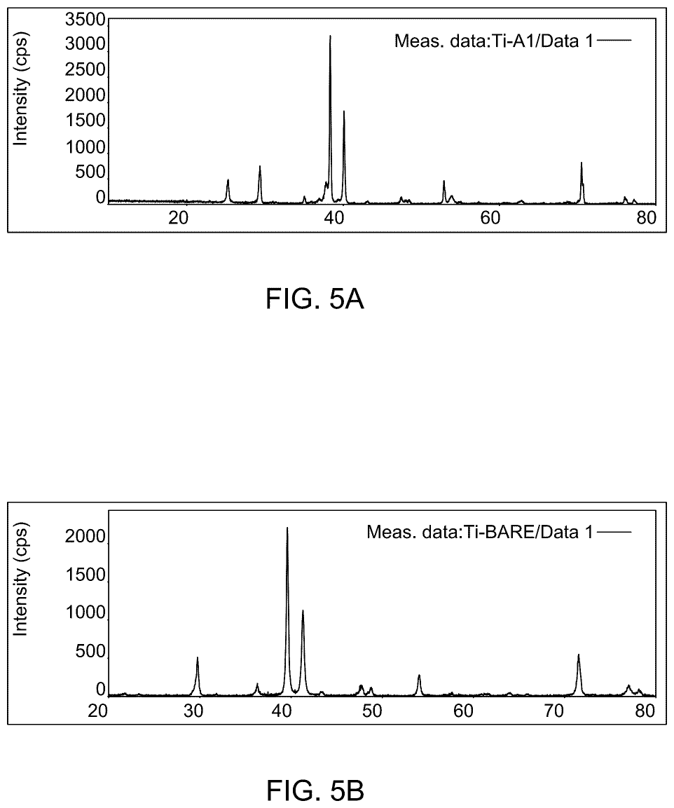

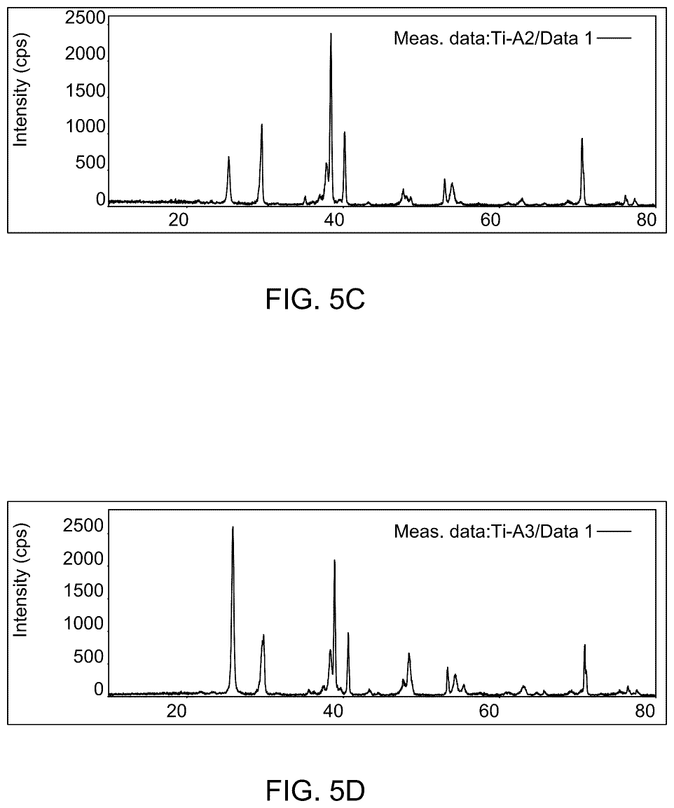

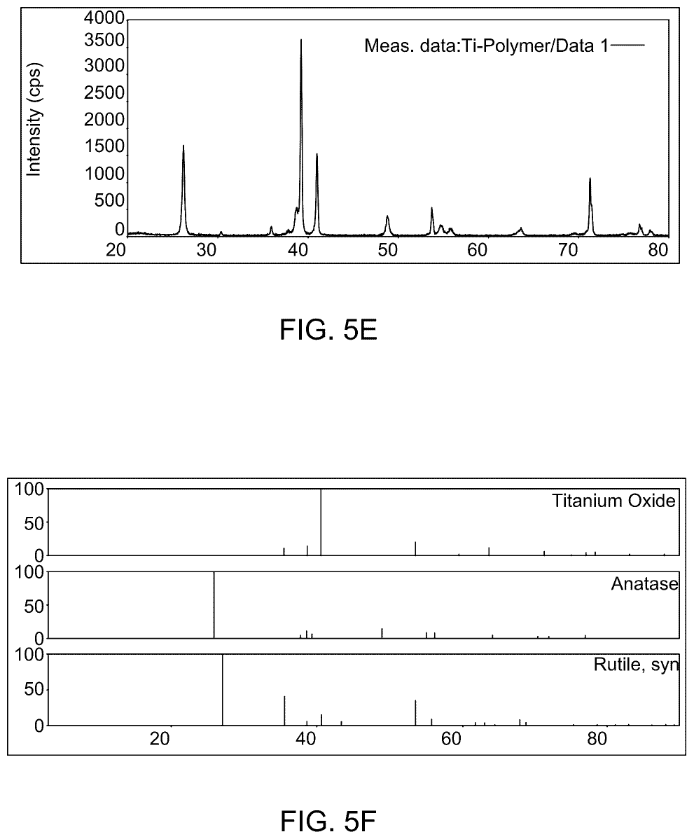

Referring to FIGS. 5A-5F, XRD analysis of bare titanium and anodized titanium dioxide nanotubes are illustrated. As a result of the anodization, the titanium dioxide nanotubes were converted to an amorphous phase. In general, an amorphous solid is formed by arranging the elements of a metal in random order. Thus, to alter the amorphous phase of the titanium dioxide nanotubes would be more suitable for reliability and consistency in biomedical applications. To achieve the crystallinity phase, particularly, the anatase phase, the titanium dioxide nanotubes are to undergo the process of annealing, and, subsequently, become more suitable for bioimplants, as described in the present disclosure. To assess the crystallinity of the titanium dioxide nanotubes, X-ray diffraction (XRD) patterns of the fabricated titanium dioxide nanotubes were monitored periodically. The obtained XRD patterns displayed in FIGS. 5A-5E indicate the phase pattern of bare titanium, the anodized sample at 40 V, 50 V, and 60 V for 30 minutes of annealing, and 50 V with PVDF, respectively. FIG. 5F depicts the intensity for TiO₂, anatase, and rutile phases. It is used as a comparison with the XRD results, as depicted in FIGS. 5A-5E. It is seen from the graphs that at all the applied voltages, the TiO₂ phase appears. In terms of the favorable anatase phase, it is noted that the anatase phase crystallinity is gradually increased as the voltage is increased.

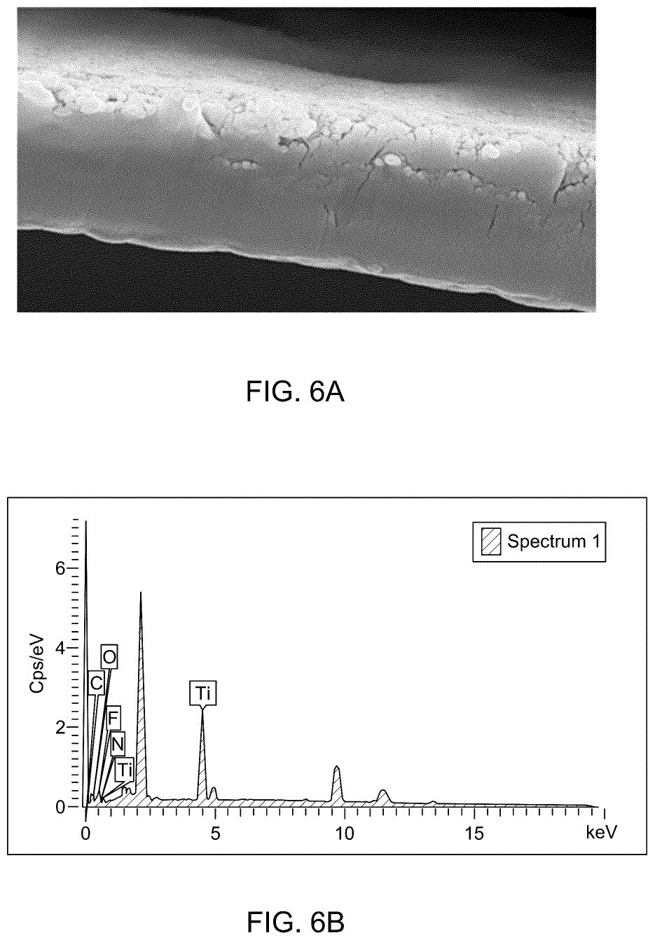

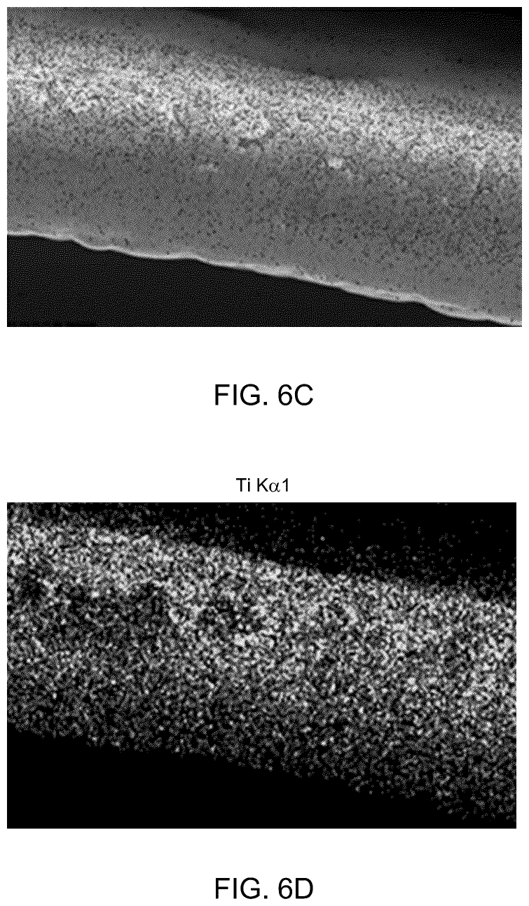

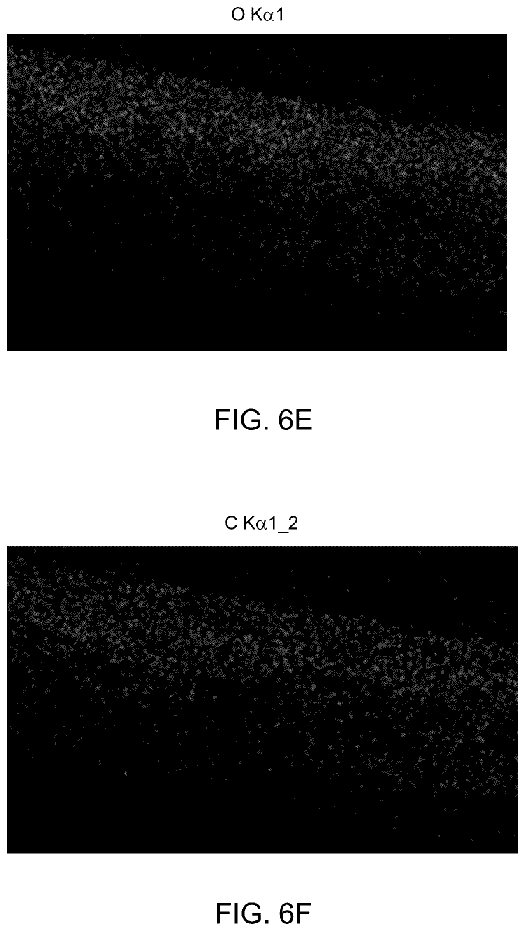

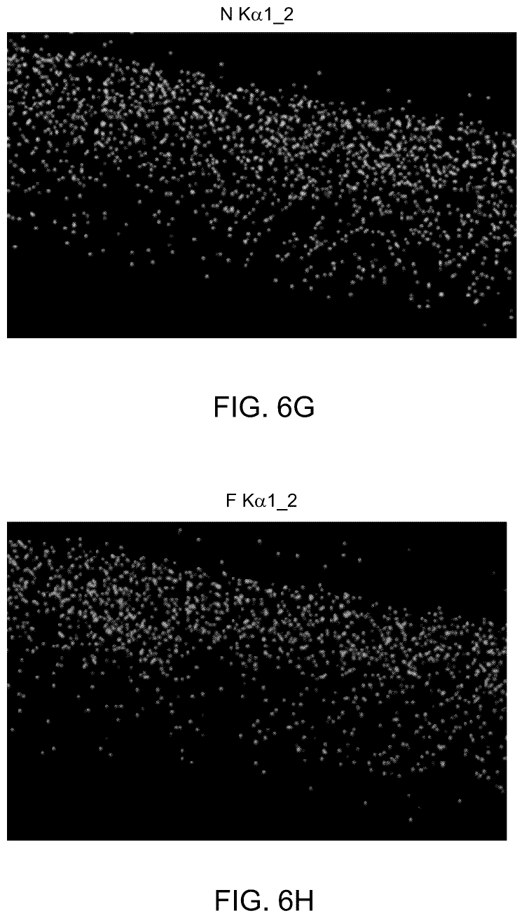

Referring to FIG. 6A, an SEM image of PVDF films deposited on the antibacterial drug-loaded titanium dioxide nanotubes, is illustrated. As can be seen from FIG. 6A, the cross-sectional SEM image depicts a thickness of 7.5 μm of the biopolymer layer deposited on the titanium dioxide nanotubes. The EDS mapping analysis results of the biopolymer-layer deposited on the titanium dioxide nanotubes indicate the presence of carbon, nitrogen, oxygen, titanium, and fluorine (FIG. 6B), confirming the deposition of PVDF films on the antibacterial drug-loaded titanium dioxide nanotubes. The EDS-layered image of the PVDF films deposited on the antibacterial drug-loaded titanium dioxide nanotubes is depicted in FIG. 6C. FIGS. 6D-6H depict the EDS-elemental mapping images for titanium, oxygen, carbon, nitrogen, and fluorine, respectively.

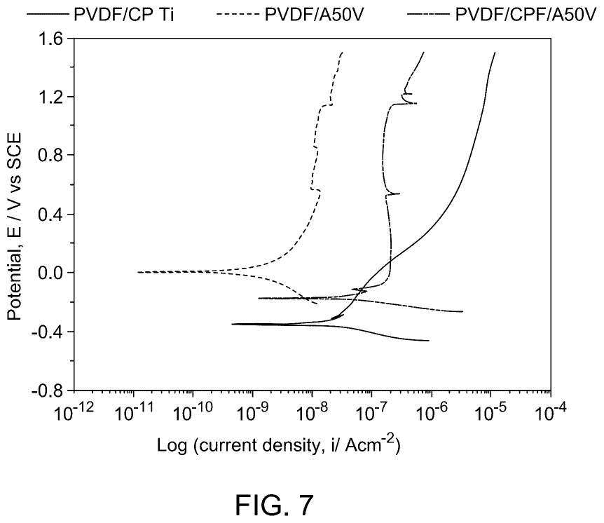

Referring to FIG. 7, PDP curves of the biopolymer-layer deposited on the titanium dioxide nanotubes loaded with antibacterial drug in simulated body fluids (SBF) medium is illustrated. To estimate the in vitro corrosion-resistant behavior of biopolymer-coated on the titanium dioxide nanotubes loaded with the antibacterial drug in SBF medium, PDP tests were performed on the titanium dioxide nanotubes, after an immersing interval of 1 hour in the SBF medium. For the sake of comparison, biopolymer-coated bare CP-Ti and biopolymer-coated titanium dioxide nanotubes without the antibacterial drug were also evaluated in the SBF medium. It is noted from the observations of the evaluation, that the biopolymer-coated titanium dioxide nanotubes with the antibacterial drug exhibited better noble corrosion voltage value and the lowest corrosion current value compared to the bare CP-Ti, validating its improved corrosion-resistant performance in SBF medium. In some embodiments, the biomedical implant system has a lower corrosion current compared to a PVDF film-coated bare Cp-Ti sample and a PVDF film-coated titanium dioxide nanotubes sample in the absence of ciprofloxacin.

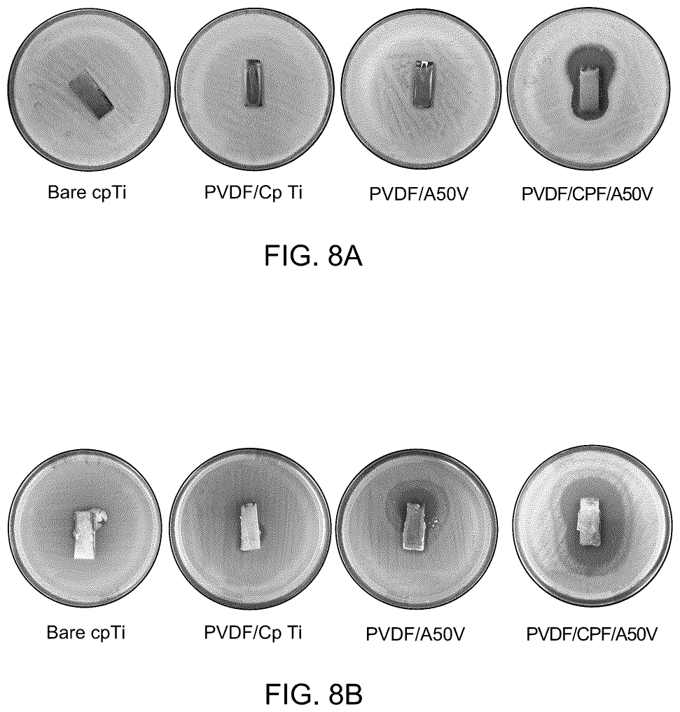

Referring to FIGS. 8A-8B, antibacterial evaluation of the biopolymer-coated titanium dioxide nanotubes loaded with the antibacterial drug is illustrated. Referring to FIG. 8A, the experiment is conducted against gram-positive bacteria. The gram-positive bacterial strain is Staphylococcus aureus, and the method used for measuring the antibacterial activity is the spread plate surface method. Referring to FIG. 8B, the experiment is conducted against gram-negative bacteria using the same spread plate surface method. The gram-negative bacterial strain is Escherichia coli. It is observed from the above-stated experiments that the quantity of bacterial colonies grown on bare Cp-Ti specimens after 24 hours of exposure was the maximum of the specimens, and the bacterial colonies on the PVDF-coated Cp-Ti specimens after 24 hours of exposure was at the lowest among the specimens. The PVDF-coated CP-Ti specimen presented a slight inhibition area against Escherichia coli and Staphylococcus aureus near the sample, indicating the inhibition of microbial growth; however, in the case of PVDF-coated titanium dioxide nanotubes loaded with ciprofloxacin, the rate of antibacterial activity was about 94.00% against Staphylococcus aureus and it was about 98.83% against Escherichia coli after 24 hours of exposure time. PVDF-coated TNT arrays loaded with ciprofloxacin exhibited the maximum antibacterial activity amongst the specimens, which may be attributed to a synergistic result of the PVDF matrix and sustained delivery of the ciprofloxacin. Overall, the rate of antibacterial activity was from 97% to 99% based on an area of microbial growth inhibition. As a result, it can be said that the biomedical implant system has a greater antibacterial resistance to Escherichia coli compared to a bare titanium sample, the PVDF-coated titanium sample, and the PVDF-coated titanium dioxide nanotubes sample in the absence of ciprofloxacin. Further, the biomedical implant system has a greater antibacterial resistance to Staphylococcus aureus compared to a bare titanium sample, the PVDF-coated titanium sample, and the PVDF-coated titanium dioxide nanotubes sample in the absence of ciprofloxacin.

In summary, polymeric coatings based on PVDF thin films, antibacterial drug-loaded titanium dioxide nanotubes were developed to improve corrosion resistance and enhance antibacterial characteristics for implant coatings for biomedical applications. The biomedical implant system of the present disclosure may find wide-ranging applications throughout the modern medical industry and specific requirements of patients and subjects with orthopedic implants.

Numerous modifications and variations of the present disclosure are possible in light of the above teachings. It is therefore to be understood that within the scope of the appended claims, the present disclosure may be practiced otherwise than as specifically described herein.

1: A biomedical implant system, comprising:
a substrate; and
a coating present on a surface of the substrate,
wherein the coating comprises,
a first layer comprising titanium dioxide nanotubes,
wherein the titanium dioxide nanotubes extend perpendicularly from a surface of the substrate,
wherein the titanium dioxide nanotubes contain ciprofloxacin,
a second layer comprising a polyvinylidene fluoride polymer,
wherein the titanium dioxide nanotubes are capped on an outer end with the polyvinylidene fluoride polymer.

2: The biomedical implant system of claim 1, wherein the first layer consists of the titanium dioxide nanotubes aligned adjacently.

3: The biomedical implant system of claim 1, wherein a first end of the titanium dioxide nanotubes is attached to the surface of the substrate and a second end of the titanium dioxide nanotubes is open-faced and exposed to the polyvinylidene fluoride polymer.

4: The biomedical implant system of claim 1, wherein the titanium dioxide nanotubes have an inside diameter of 20 nm to 60 nm.

5: The biomedical implant system of claim 1, wherein the titanium dioxide nanotubes have a length of 1 μm to 5 μm.

6: The biomedical implant system of claim 1, wherein the titanium dioxide nanotubes have a wall thickness of 0.2 nm to 5 nm.

7: The biomedical implant system of claim 1, wherein the second layer has a thickness of 5 μm to 10 μm.

8: The biomedical implant system of claim 1, wherein the second layer has a thickness of 5 μm to 10 μm and is formed of a mixture of polyvinylidene fluoride and polylactic acid in a mass ratio of 70-95:5-30 such that up to 10% by number of the titanium dioxide nanotubes are capped on an outer end with the polylactic acid and the remaining titanium dioxide nanotubes are capped on an outer end with polyvinylidene fluoride polymer.

9: The biomedical implant system of claim 1, wherein the substrate is a commercially pure titanium sample.

10: The biomedical implant system of claim 1, wherein the system is made by a process comprising:
anodizing the substrate in a solution to form the first layer of the coating,
wherein the solution comprises an organic solvent, water, and an inorganic compound,
wherein the anodizing is at a voltage of 20 V to 80 V for a time of 10 to 200 minutes,
drying the first layer of the coating;
annealing the first layer of the coating;
loading the first layer of the coating with ciprofloxacin; and
covering the first layer of the coating with the second layer of the coating.

11: The biomedical implant system of claim 10, wherein the anodizing occurs in a two-electrode cell with the substrate for an anode and a graphite rod for a cathode.

12: The biomedical implant system of claim 10, wherein the loading is done in a polar organic solvent.

13: The biomedical implant system of claim 10, wherein the ciprofloxacin is loaded into the titanium dioxide nanotubes at a concentration of 0.01 M to 0.10 M.

14: The biomedical implant system of claim 1, wherein the biomedical implant system comprises titanium, oxygen, carbon, nitrogen, and fluorine.

15: The biomedical implant system of claim 1, wherein 70 to 95 percent of titanium dioxide in the titanium dioxide nanotubes have an anatase phase crystallinity.

16: The biomedical implant system of claim 1, wherein the biomedical implant system has a lower corrosion current compared to a polyvinylidene fluoride polymer film coated titanium sample and a polyvinylidene fluoride polymer film coated titanium dioxide nanotube sample in the absence of ciprofloxacin.

17: The biomedical implant system of claim 1, wherein the biomedical implant system has a greater antibacterial resistance to Escherichia coli compared to a bare titanium sample, the polyvinylidene fluoride polymer film coated titanium sample, and the polyvinylidene fluoride polymer film coated titanium dioxide nanotube sample in the absence of ciprofloxacin.

18: The biomedical implant system of claim 17, wherein a rate of antibacterial activity is from 97 to 99 percent based on an area of microbial growth inhibition.

19: The biomedical implant system of claim 1, wherein the biomedical implant system has a greater antibacterial resistance to Staphylococcus aureus compared to the bare titanium sample, the polyvinylidene fluoride polymer film coated titanium sample, and the polyvinylidene fluoride polymer film coated titanium dioxide nanotube sample in the absence of ciprofloxacin.

20: The biomedical implant system of claim 1, wherein the ciprofloxacin is released through the polyvinylidene fluoride polymer over time.
